(19) United States
(12) Patent Application Publication
(10) Pub. No.: US 2026/0306451 A1
(43) Pub. Date: Oct. 8, 2026

(54) METHODS FOR USING BACTERIA AND HOST/MICROBIOTA-DERIVED METABOLITES FOR DIAGNOSIS AND TREATMENT OF ENDOMETRIOSIS

(71) Applicant: Baylor College of Medicine, Houston, TX (US)

(72) Inventor: Ramakrishna Kommagani, Houston, TX (US)

(73) Assignee: Baylor College of Medicine, Houston, TX (US)

(21) Appl. No.: 19/481,269

(22) PCT Filed: May 3, 2024
(86) PCT No.: PCT/US2024/027820

§ 371 Date: Nov. 4, 2025

Related U.S. Application Data
(60) Provisional application No. 63/500,476, filed on May 5, 2023.

(51) Int. Cl. A61K 31/404; A61P 15/00; C12Q 1/04; G01N 33/68

(52) U.S. Cl. CPC A61K 31/404; A61P 15/00; C12Q 1/04; G01N 33/689; G01N 2800/364

(57) ABSTRACT

Compositions and methods involving bacteria and metabolites derived from the microbiotaare provided herein in certain aspects to reduced endometriosis. This includes therapeutic compositions comprising the bacteria and/or metabolites, as well as methods of administering the bacteria and/or metabolites to an individual in need thereof.

### CROSS-REFERENCE TO RELATED APPLICATIONS

This application claims priority to U.S. Provisional Application No. 63/500,476 filed May 5, 2023, all of which is incorporated by reference in its entirety.

This invention was made with government support under HD10268 awarded by the National Institutes of Health. The government has certain rights in the invention.

### BACKGROUND

### I. Field of the Disclosure

This disclosure relates to the field of microbiology, oncology, and medicine.

### II. Background

Endometriosis is a debilitating health condition prevalent in reproductive age women, and that develops when endometrium (tissue lining the uterus) grows outside the uterus in some women due to reasons currently not understood. Inflammation resulting from these growing implants closely associates with disease severity and is aggravated by ‘leaky gut’ and has been linked to inflammatory bowel disorders. Several studies have addressed the concerns on perturbed gut microbiota profiles in endometriosis. However, no study reports the gut metabolome that mediate microbe-microbe and host-microbe interactions for pathogenesis.

### BRIEF SUMMARY

Compositions and methods involving metabolites and/or bacteria are provided herein. This includes therapeutic compositions comprising the metabolites and/or bacteria as well as methods of administering the metabolites and/or bacteria to an individual in need thereof.

Certain aspects concern methods of treating a disease in an individual. The disease can be endometriosis. The disease can comprise endometrium growth outside of the uterus. The disease can comprise aberrant growth of endometrial epithelial cells. Also disclosed are methods of inhibiting endometriotic epithelial cells, methods of preventing endometriosis, and methods of preventing endometriotic epithelial cell growth. In some aspects, the method comprises the step of administering to the individual an effective amount of an indole metabolite. In some aspects, the method comprises the step of administering to the individual an effective amount of 4-hydroxyindole (4HI) and/or indole-4-carbaldehyde. In some aspects, the method comprises the step of administering to the individual an effective amount of 4HI. In some aspects, the method comprises the step of administering to the individual an effective amount of indole-4-carbaldehyde. The 4HI and indole-4-carbaldehyde may be administered concurrently or sequentially. In some aspects, the method comprises the step of administering to the individual an effective amount of any therapeutic composition described herein. In some aspects, the method comprises the step of administering to the individual an effective amount of at least metabolite of Table 1 and/or at least one bacteria of Table 2. In some aspects, the individual is administered a composition comprising any metabolite described herein and any bacteria described herein. The metabolite(s) may be administered concurrently with the bacteria. The metabolite(s) may be administered sequentially with the bacteria. In certain aspects, the method further comprises administering an additional therapeutic composition to the individual. The additional therapeutic composition may be administered before, during, or after administration of other therapeutic compositions, including the metabolite and/or bacterial compositions.

In certain aspects, the individual is determined to have endometriosis after one or more metabolites and/or at least one bacteria is measured in a biological sample from the individual. The one or more metabolites may be an indole metabolite, such as 4-hydroxyindole (4HI) and/or indole-4-carbaldehyde. The metabolite may be any metabolite of Table 1. The bacteria may be a bacteria of any genus, family or order of Table 2. The biological sample may be any sample useful for determining the metabolites and/or bacteria, such as a stool sample or biopsy.

Also disclosed are therapeutic compositions comprising any metabolite and/or bacteria disclosed herein. In some aspects, the therapeutic composition comprises an indole metabolite. In some aspects, the therapeutic composition comprises 4HI and/or indole-4-carbaldehyde. In some aspects, the therapeutic composition comprises a metabolite of Table 1 and/or a bacteria of Table 2.

Also disclosed are methods for detecting a disease, including detecting endometriosis, in an individual. Also disclosed are methods for detecting aberrant endometrial growth in an individual. In some aspects, the method comprises measuring one or more metabolites from Table 1 and/or at least one bacteria from Table 2 in a biological sample from the individual. In some aspects, the method comprises measuring 4HI and/or indole-4-carbaldehyde in a biological sample. The biological sample can be any sample, including a stool sample or biopsy. In some aspects, the measured metabolites are compared to a standard. In some aspects, the standard comprises measurements from a healthy individual. In some aspects, the standard comprises a biological sample from a healthy individual. The healthy individual may be an individual known to not have, or not diagnosed with, endometriosis. The measurement from the healthy individual may be a measurement of the one or more metabolites from Table 1 and/or at least one bacteria from Table 2 in a biological sample from the healthy individual. The methods of detecting may further comprise administering a therapeutic composition, including any therapeutic composition disclosed herein such as any metabolite and/or bacterial composition disclosed herein, to the individual. The therapeutic compositions may be administered in any order and in any manner.

Also disclosed are methods for assaying a microbiome profile of an individual. The method can comprise assaying a stool sample from the individual. The method can comprise measuring one or more metabolites of Table 1 and/or one or more bacteria of Table 2. The method can comprise one or more steps disclosed herein. Also disclosed are methods for diagnosing an individual with endometriosis. The method can comprise measuring one or more metabolites disclosed herein and/or one or more bacteria disclosed herein. The diagnostic method can be used to determine whether an individual has or does not have endometriosis. In some aspects, the method comprises determining the individual has endometriosis based on the measured metabolites and/or bacteria. In some aspects, the method comprises determining the severity of the endometriosis based on the measured metabolites and/or bacteria. The method can be used to determine a treatment for the individual. In some aspects, the method comprises determining a therapeutic strategy and/or administering a therapeutic composition to the individual based on the measured metabolites and/or bacteria.

Also disclosed are method of measuring one or more metabolites and/or one or more bacteria in a sample from an individual. The metabolites may be any metabolites described herein, including any metabolite of Table 1. The bacteria may be any bacteria described herein, including any metabolite of Table 2.

Any method in the context of a therapeutic, diagnostic, or physiologic purpose or effect may also be described in “use” claim language such as “Use of” any compound, composition, or agent discussed herein for achieving or implementing a described therapeutic, diagnostic, or physiologic purpose or effect.

Use of the one or more sequences or compositions may be employed based on any of the methods described herein. Other aspects and embodiments are discussed throughout this application. Any embodiment or aspect discussed with respect to one aspect of the disclosure applies to other aspects of the disclosure as well and vice versa.

It is specifically contemplated that any limitation discussed with respect to one embodiment or aspect of the disclosure may apply to any other embodiment or aspect of the disclosure. Furthermore, any composition of the disclosure may be used in any method of the disclosure, and any method of the disclosure may be used to produce or to utilize any composition of the disclosure. Aspects of an embodiment set forth in the Examples are also embodiments that may be implemented in the context of embodiments discussed elsewhere in a different Example or elsewhere in the application, such as in the Summary of Disclosure, Detailed Description, Claims, and description of drawing.

Other objects, features and advantages of the present disclosure will become apparent from the following detailed description. It should be understood, however, that the detailed description and the specific examples, while indicating specific aspects of the disclosure, are given by way of illustration only, since various changes and modifications within the spirit and scope of the disclosure will become apparent to those skilled in the art from this detailed description.

### BRIEF DESCRIPTION OF THE DRAWINGS

The following drawings form part of the present specification and are included to further demonstrate certain aspects of the present disclosure. The disclosure may be better understood by reference to one or more of these drawings in combination with the detailed description of specific aspects presented herein.

FIGS. 1A-1F show untargeted metabolomics analyses. metabolomics and comparative profiles from altered metabolites and functions in endometriosis and healthy groups (A) Classes enriched in the total metabolites (n=371) detected in Healthy and EMS groups denoted by different color dots. Size of the dots represent the enrichment ratio; color denotes the lowest (yellow) to highest (red) p-values for each class of metabolites. (B) Partial Least Squares-Discriminant Analysis (PLS-DA) scores are indicated by different color circles for Healthy and EMS groups (C) Volcano plots of all identified metabolites (n=371) from untargeted metabolomics. The x-axis indicates Log 2 (fold change) while the y-axis indicates-Log 10 (P-value). Every single metabolite is represented as a dot. Different colors were used to represent down-regulated (red), up-regulated (green), or non-significant (black) metabolites. (E) Heatmap based on significantly altered metabolites (n=71) altered significantly (FDR Adjusted p-value <0.05) in between Healthy and EMS groups. (F) Enriched functional pathways detected in the total metabolites data. Each dot denotes a functional pathway colored and sized based on p-values (lowest red to highest yellow) and enrichment ratio, respectively.

FIGS. 2A-2D show gut microbiome profiles and correlation-based integration of metabolomes. (A) (B) Beta diversity compared between Healthy and EMS groups using Principal Component Analysis (PCoA) on weighted UniFrac distances. The centroids and dispersion of the groups are shown using thin colored lines (from each sample point to corresponding centroid). The plot is supplemented with PERMANOVA statistics for the group factor. (C) The relative taxonomic abundance of bacterial genera in the two groups. Significantly altered taxa are denoted with red asterisk. (D) Spearman correlation between metabolites and microbial genera. Only the metabolites of microbiota origin that correlated significantly (p<0.05) are shown. The metabolites that are altered (FC >1) in endometriosis are denoted with asterisk. (E) Violin plots of the significantly altered metabolites from microbial driven indole pathway in endometriosis-4-hydroxyindole (4HI) and indole-4-carbaldehyde (I4CAld) with fold change, FC >1. (F) Selected bacteria-metabolite correlation pattern in endometriosis. The shading of the squares indicates the correlation (green color metabolite denotes positive correlation; red colored metabolite denotes negative correlation). qPCR-based quantitative estimation of indicated bacterial taxon using 16S rDNA gene amplification products from control and patient cohorts (n=10 samples each). Statistical significance was determined using unpaired t-test with Welch's correction (* denotes p-value <0.05, *** denote p-value <0.001). (G) Heatmap of metabolites.

FIGS. 3A-3D show enriched disease signatures and functional pathways in gut metabolome. (A) Bar graph showing the top enriched disease signatures within the altered metabolites (n=91-bowel issues Healthy 2). (B) Bar chart showing the number of metabolites covered by each enriched disease signature. The x-axis indicates the number of assigned metabolites to total metabolites while the y-axis indicates the disease signature. Each bar is color coded for p-value <0.05: Red: low; yellow: high. (C) The microbial metabolism of Tryptophan (Trp) showing serotonin and indole pathways. (D) Altered metabolites belonging to Indole pathway determined in the metabolomics data.

FIGS. 4A-4C show treatment with indole metabolites in vitro and in vivo. MTT cell viability assays of iHEECs/Luc treated with different concentrations of (A) indole (B) 4-hydroxyindole (C) indole-4-carbaldehyde. Results are shown as mean±SE (n=3) and experiment was repeated three times.

FIGS. 5A-C show 4HI and 14CAld inhibit the initiation and progression of endometriosis in vivo. (A) Schematic of experimental timeline and procedures in mouse model of endometriosis employed for testing suppressive effect of 4-hydroxyindole (4HI) and indole-4 carbaldehyde (I4CAld) on disease establishment. (B) Representative images of ectopic endometriotic lesions from the vehicle, 4HI, I4CAld and indole treated groups, 14 days after induction of endometriosis. Lesion numbers, volumes, and masses of ectopic endometriotic lesions from the indicated treatment groups are shown. Data are presented as mean±SE (n=5). *Indicates significance between groups, *p-value <0.05; **p<. 01 (C) Representative hematoxylin and eosin (H&E) stained sections of lesions and sections stained with anti-Ki67 (proliferation) and anti-F4/80 (macrophages and inflammation). Scale bar=50 μm. E, epithelium; S, stroma; G, glands; GE, glandular epithelium.

FIG. 6 shows identification of biomarker metabolites in stool samples from endometriosis patients. ROC curve performance of biomarkers (AUC>0.8) determined from metabolites data from stool samples from endometriosis patients, namely, Perfluoro-1-butanesulfonic acid (PFBS), 1,5-dimethyl-4-[2-(3-methyl-5-oxo-1-phenyl-4,5-dihydro-1H-pyrazol-4-yliden) hydrazino]-2-phenyl-2,3-dihydro-1H-pyrazol-3-one, 4-Dodecylbenzenesulfonic acid, Niacinamide, Linoleic acid, 2′-Deoxyadenosine, N-Formyl-L-methionine, 2-Furyl(5-hydroxy-1-benzofuran-3-yl) methanone, NP-014113, Adenine, Cytosine and Adenosine.

FIGS. 7A-7C show indole-derived metabolites 4HI and I4CAld are lower in stools of women with endometriosis. (A) Pathway showing microbial production of indole and its derivatives. (B) Distinct altered derivatives of indole that deviate non-significantly in endometriosis (FC=Fold Change). (C) Box plots showing upward-, downward- or unchanged trends of selected metabolites from LC-MS validation studies of the selected metabolites.

FIGS. 8A-8B show 4HI and I4CAld reduces the endometriosis-associated inflammation (A) and pain (B). (A) Flow cytometric dot plots and histogram overlay for cell sorting on the peritoneal fluid at day 14 using the Cytek Aurora. The MHCII⁺ CD11b⁺ F4/80 hi mac (M1 like macrophage) and CD206⁺ CD11b⁺ F4/80 hi mac (M2 like Macrophage) in indicated groups are shown. (B) Assessment of endometriosis-associated hyperalgesia through hind paw withdrawal-based Von Frey test on Days 0 and 14 after endometriosis induction in mice. Data are presented as mean±SE (n=4). *Indicates significance between groups, **p-value <0.05.

FIGS. 9A-9C show 4HI and I4CAld regressed the established lesions in mouse induced with endometriosis. (A) Schematic of experimental timeline and procedures employed for testing therapeutic action of 4-hydroxyindole (4HI), indole-4 carbaldehyde (I4CAld) and indole on regression of established endometriotic lesions in heterologous mouse model of the disease. (B) Representative images of ectopic endometriotic lesions from the vehicle and metabolite treated groups, 28 days after induction of endometriosis. Lesion numbers, volumes and masses of ectopic endometriotic lesions from the indicated treatment groups are shown. Data are presented as mean±SE (n=5). *Indicates significance between groups, *p-value <0.05, **p<. 01, ***p<0.001. (C) Representative hematoxylin and eosin (H&E) stained- and immunofluorescence in sections of lesions derived from mice induced with endometriosis. The sections were stained with anti-Ki67 (proliferation) and anti-F4/80 (macrophages and inflammation). Scale bar=50 μm. E, epithelium; S, stroma; G, glands; GE, glandular epithelium.

FIGS. 10A-10D show 4HI and I4CAld regressed the established lesions in mice xenografted with human endometriotic cells. (A) Schematic of experimental timeline and procedures employed for testing therapeutic action of 4-hydroxyindole (4HI), indole-4 carbaldehyde (I4CAld) and indole on regression of established endometriotic lesions in mice induced with endometriosis using human endometriotic cells in xenograft model of the disease. (B) Representative bioluminescence images of lesions from mice transplanted with human endometriotic cells at indicated days and lesion data collected on day 28 from indicated groups. (C) Representative images of ectopic endometriotic lesions from the vehicle and metabolite treated groups, 28 days after induction of endometriosis. Lesion numbers, volumes and masses of ectopic endometriotic lesions from the indicated treatment groups are shown. Data are presented as mean±SE (n=5). *Indicates significance between groups, *p-value <0.05, **p<. 01, ***p<0.001. (D) Representative hematoxylin and eosin (H&E) stained- and immunofluorescence in sections of lesions derived from mice induced with human endometriotic cells. The sections were stained with anti-Ki67 (proliferation) and anti-F4/80 (macrophages and inflammation). Scale bar=50 μm. E, epithelium; S, stroma; G, glands; GE, glandular epithelium.

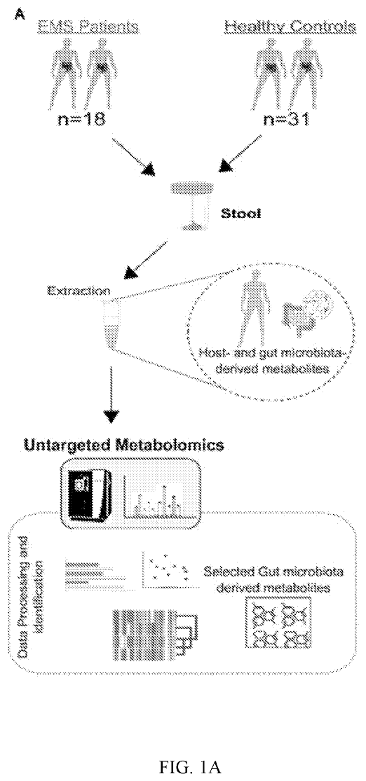

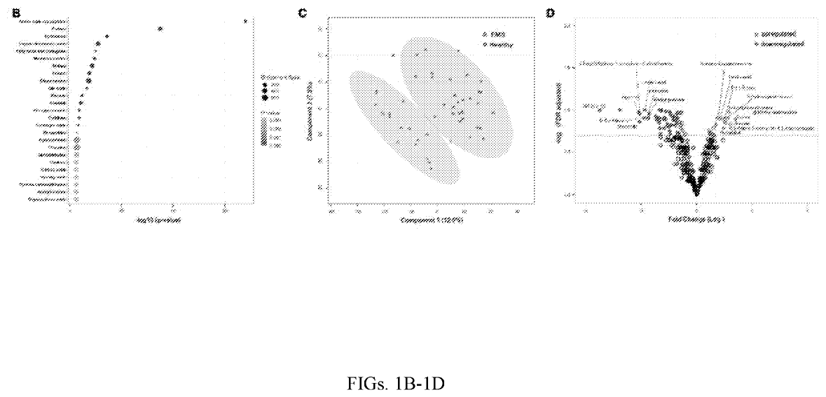

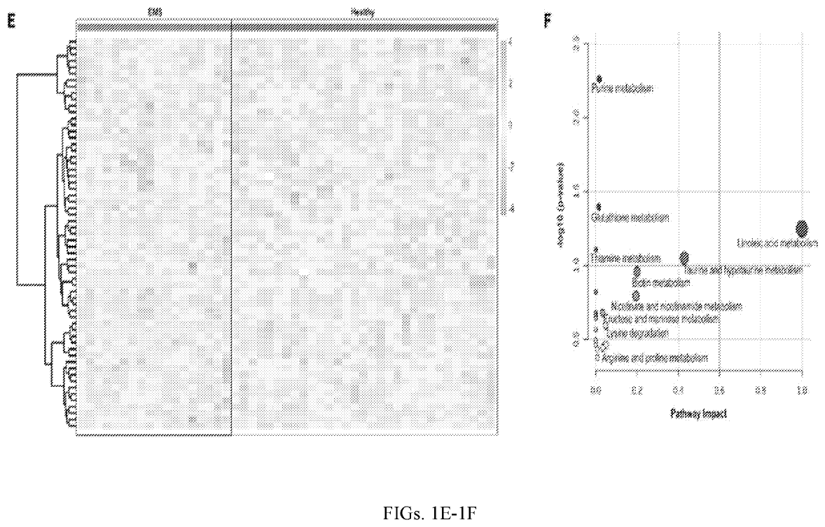

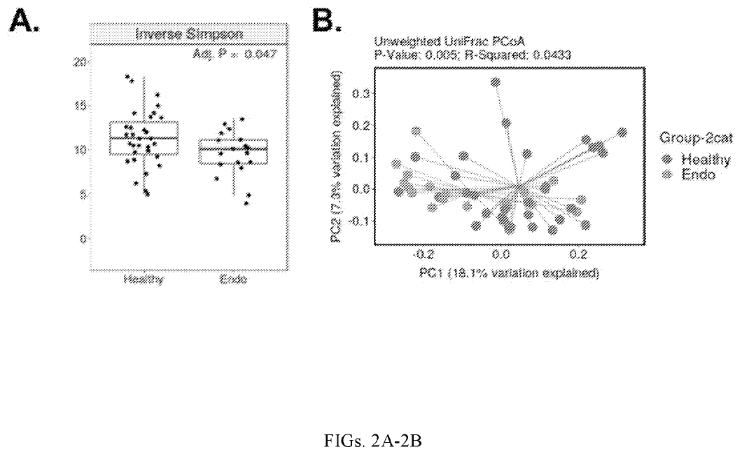

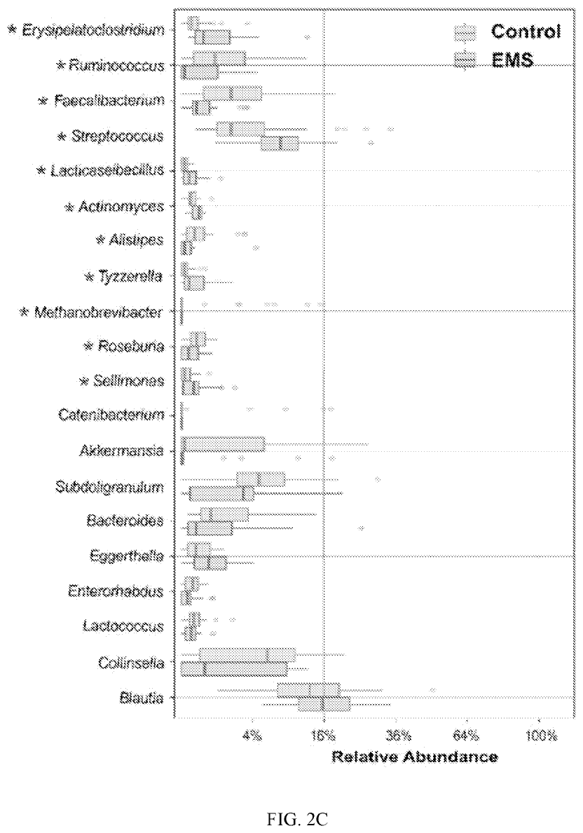

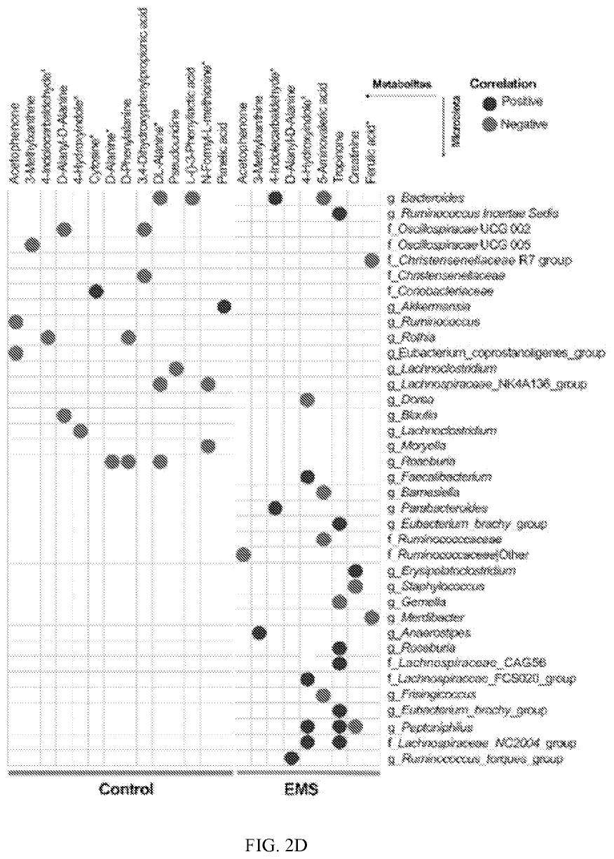

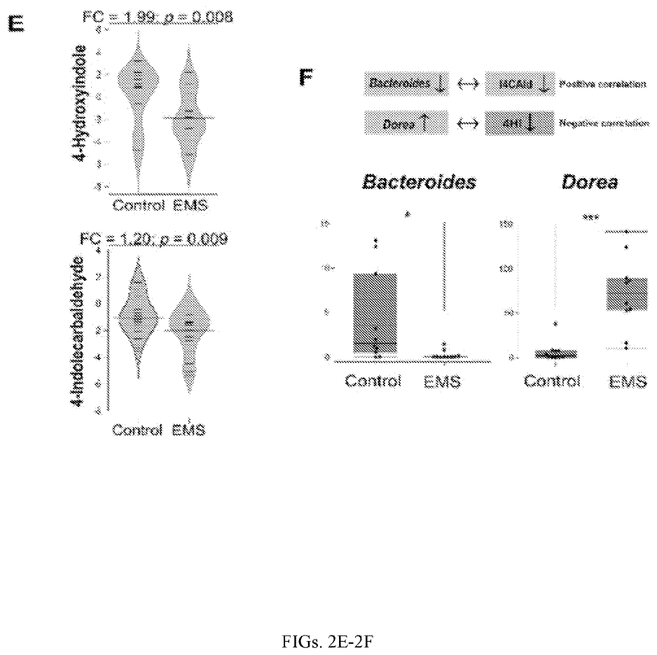

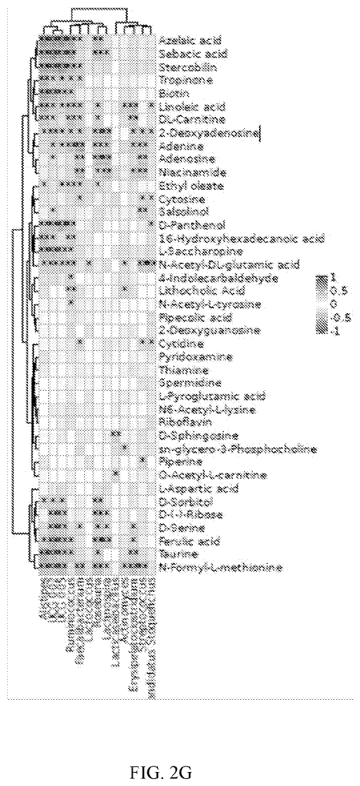

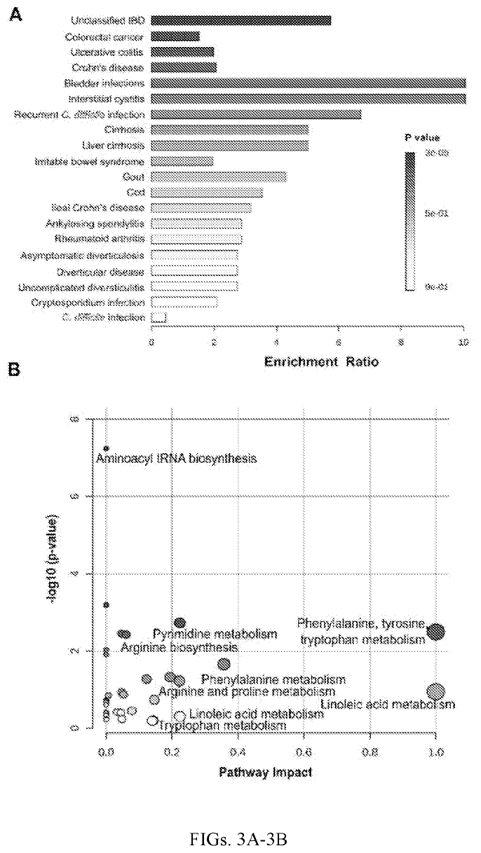

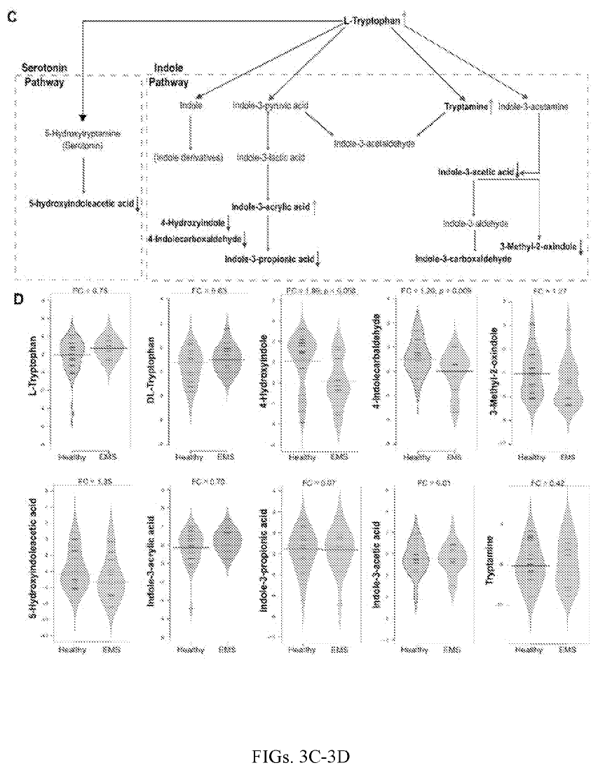

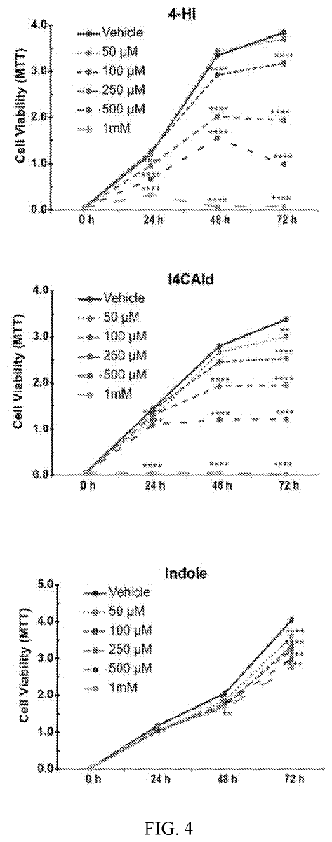

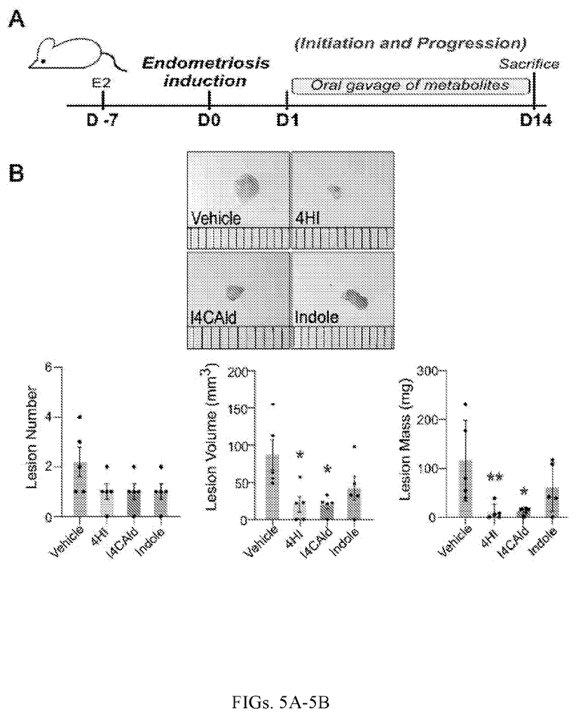

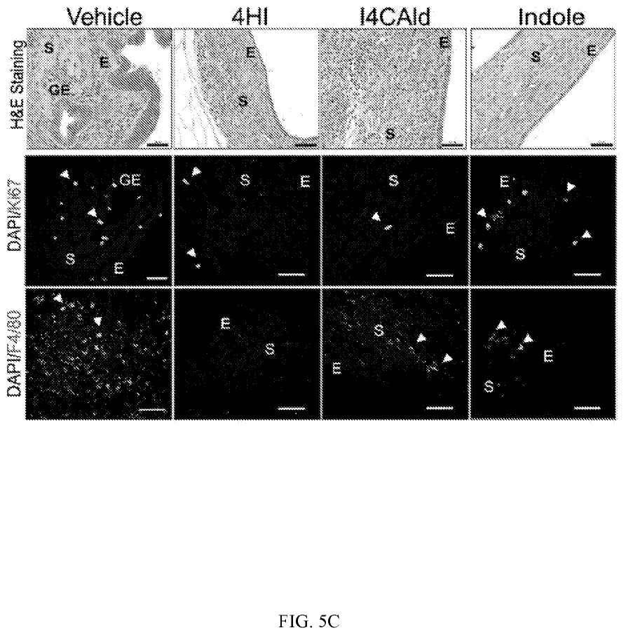

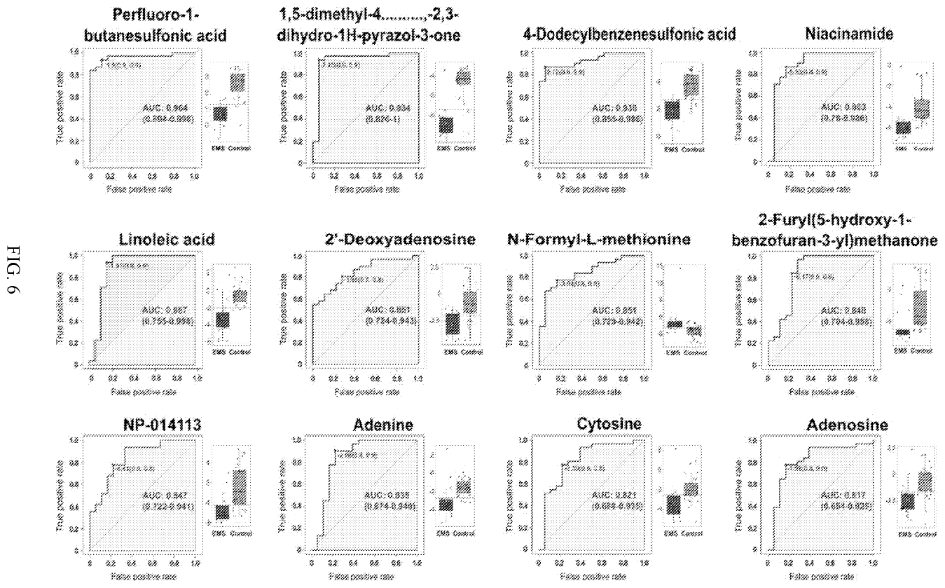

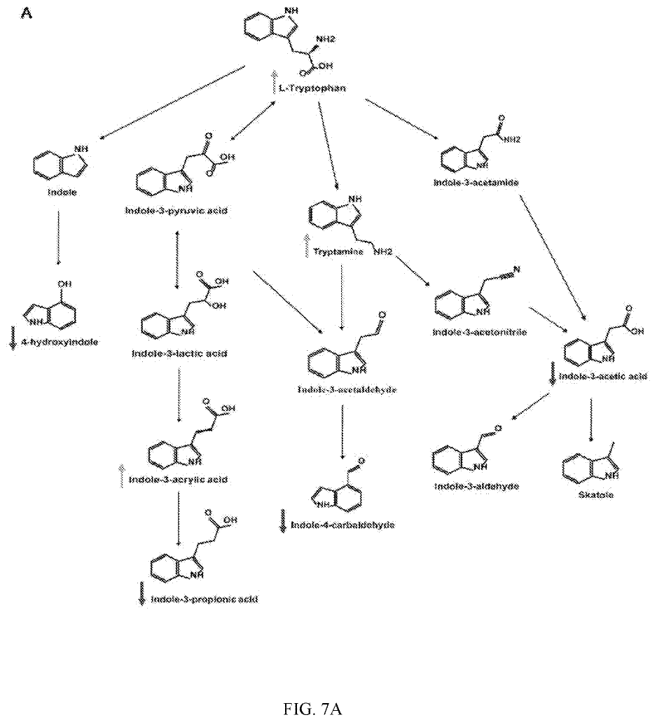

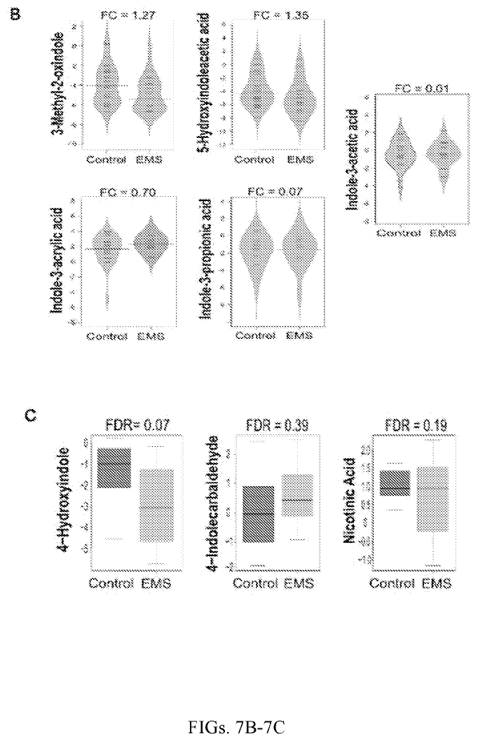

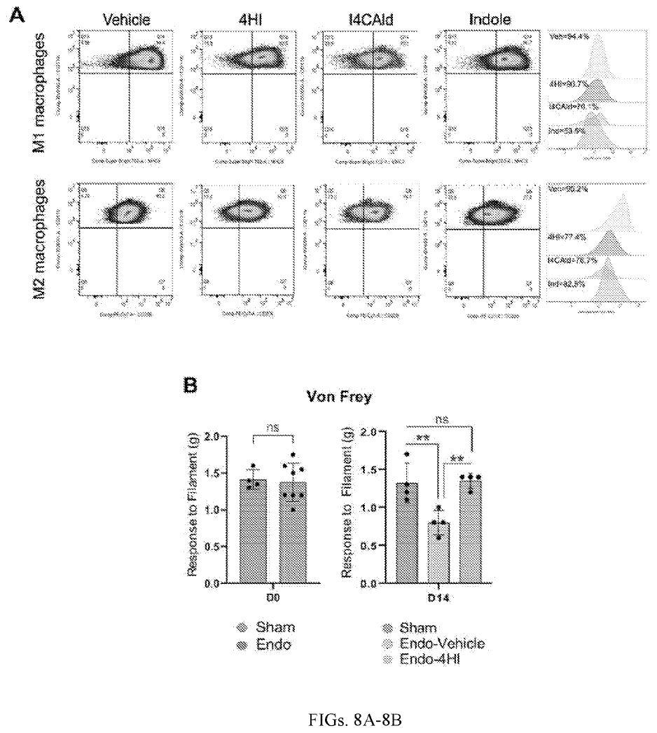

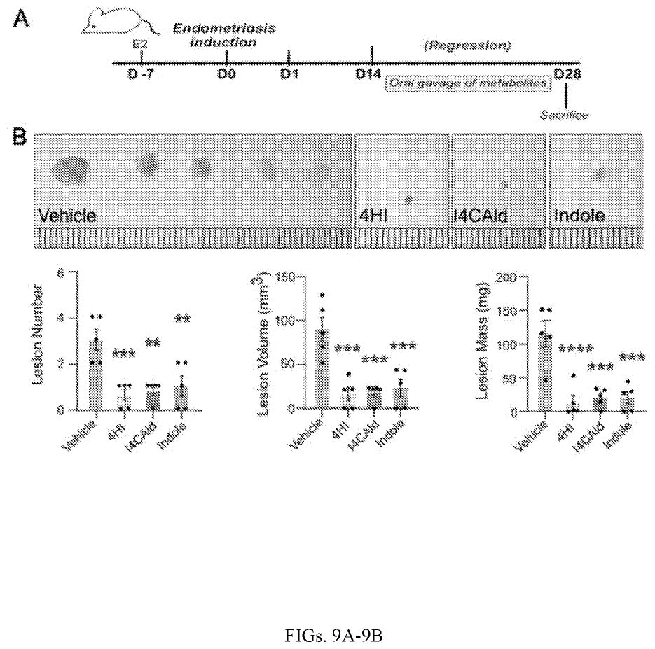

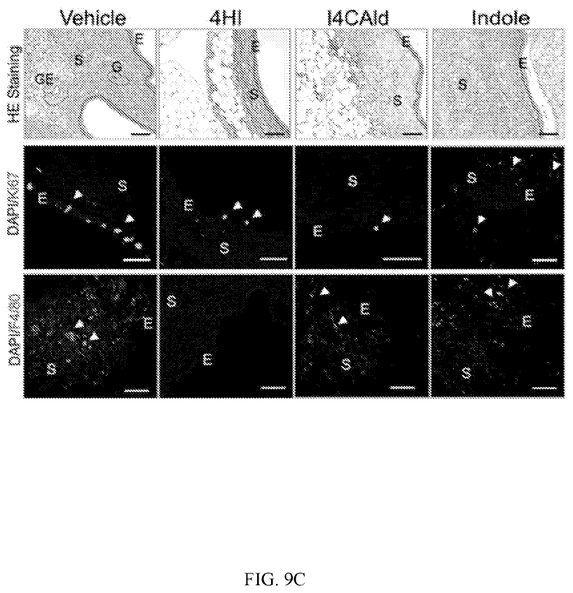

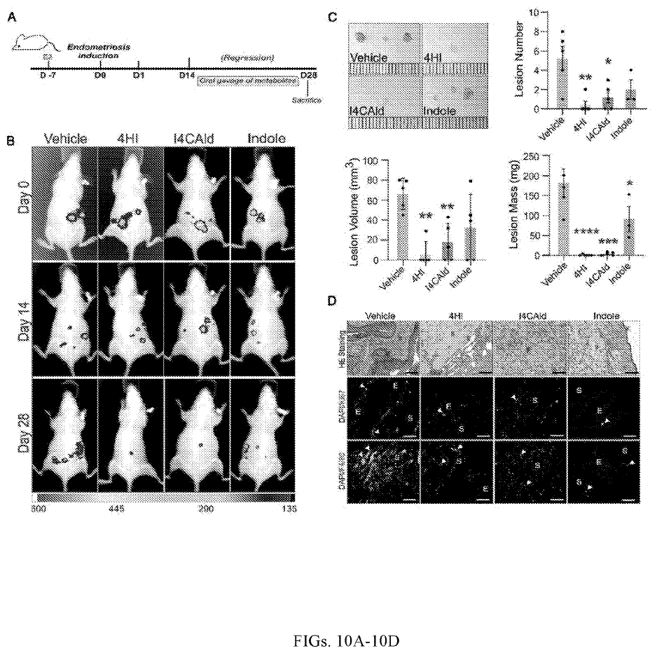

### DETAILED DESCRIPTION

### I. Definitions

Throughout this application, the term “about” is used according to its plain and ordinary meaning in the area of cell and molecular biology to indicate that a value includes the standard deviation of error for the device or method being employed to determine the value.

The use of the word “a” or “an” when used in conjunction with the term “comprising” may mean “one,” but it is also consistent with the meaning of “one or more,” “at least one,” and “one or more than one.” Any term used in singular form also comprise plural form and vice versa.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment or aspect.

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The compositions and methods for their use can “comprise,” “consist essentially of,” or “consist of” any of the ingredients or steps disclosed throughout the specification. The phrase “consisting of” excludes any element, step, or ingredient not specified. The phrase “consisting essentially of” limits the scope of described subject matter to the specified materials or steps and those that do not materially affect its basic and novel characteristics. It is contemplated that embodiments and aspects described in the context of the term “comprising” may also be implemented in the context of the term “consisting of” or “consisting essentially of.”

The term “isolated” encompasses a bacterium or other entity or substance that has been (1) separated from at least some of the components with which it was associated when initially produced (whether in nature or in an experimental setting), and/or (2) produced, prepared, purified, and/or manufactured by the hand of man. Isolated bacteria may be separated from at least about 10%, about 20%, about 30%, about 40%, about 50%, about 60%, about 70%, about 80%, about 90%, or any range derivable therein, or more of the other components with which they were initially associated. In some aspects, isolated bacteria are more than about 80%, about 85%, about 90%, about 91%, about 92%, about 93%, about 94%, about 95%, about 96%, about 97%, about 98%, about 99%, or any range derivable therein, or more than about 99% pure. As used herein, a substance is “pure” if it is substantially free of other components.

The terms “purify,” “purifying” and “purified” refer to a bacterium or other material that has been separated from at least some of the components with which it was associated either when initially produced or generated (e.g., whether in nature or in an experimental setting), or during any time after its initial production. A bacterium or a bacterial population may be considered purified if it is isolated at or after production, such as from a material or environment containing the bacterium or bacterial population, and a purified bacterium or bacterial population may contain other materials up to about 10%, about 20%, about 30%, about 40%, about 50%, about 60%, about 70%, about 80%, about 90%, or any range derivable therein, or above about 90% and still be considered “isolated.” In some aspects, purified bacteria and bacterial populations are more than about 80%, about 85%, about 90%, about 91%, about 92%, about 93%, about 94%, about 95%, about 96%, about 97%, about 98%, about 99%, or any range derivable therein, or more than about 99% pure. In the instance of bacterial compositions provided herein, the one or more bacterial types present in the composition can be independently purified from one or more other bacteria produced and/or present in the material or environment containing the bacterial type. Bacterial compositions and the bacterial components thereof are generally purified from residual habitat products.

An effective amount of the pharmaceutical composition is determined based on the intended goal. The term “unit dose” or “unit dosage” refers to physically discrete units suitable for use in a subject, each unit containing a predetermined-quantity of the pharmaceutical composition calculated to produce the desired responses discussed above in association with its administration, i.e., the appropriate route and treatment regimen. The quantity to be administered, both according to number of treatments and unit dose, depends on the protection or effect desired.

It is contemplated that any aspect discussed in this specification can be implemented with respect to any method or composition of the disclosure, and vice versa. Furthermore, compositions of the disclosure can be used to achieve methods of the disclosure.

### II. Bacteria and Metabolites

Disclosed herein are bacteria and microbiota-derived metabolites that may be useful for treating and/or preventing endometriosis. The disclosed bacteria and/or metabolites may also be useful for detecting and/or diagnosing endometriosis by measuring the levels of one or more of the bacteria and/or one or more of the metabolites in a biological sample. Also disclosed are compositions comprising the bacteria and microbiota-derived metabolites. The bacteria and/or metabolites useful for treating and/or preventing endometriosis may include any bacteria and/or metabolites described herein, including those discovered by the methods disclosed herein.

The disclosed metabolites include indole metabolites, such as 4-hydroxyindole (4HI) and/or indole-4-carbaldehyde. Also disclosed are compositions comprising 4HI and/or indole-4-carbaldehyde. The disclosed microbiota-derived metabolites include the metabolites of Table 1. Also disclosed are compositions comprising one or more metabolites of Table 1.

TABLE 1
Metabolites

Ferulic acid
D-(−)-Ribose
N-Acetyl-D-galactosamine
D-Serine
N-Formyl-L-methionine
Methyl acetoacetate
DL-Alanine
D-Sorbitol
6-Hydroxypicolinic acid
6-Chloro-5-methyl-1H-1,2,3-benzotriazole
Taurine
sn-glycero-3-Phosphocholine
Niacinamide
Adenosine
Adenine
2′-Deoxyadenosine
Cytosine
Salsolinol
Cytidine
Prolylleucine
2-(2-amino-3-methylbutanamido)-3-phenylpropanoic acid
Spermidine
2-Deoxyguanosine
Anhydroecgonine
Perfluoro-1-butanesulfonic acid (PFBS)
1,5-dimethyl-4-[2-(3-methyl-5-oxo-1-phenyl-4,5-dihydro-1H-
pyrazol-4-yliden)hydrazino]-2-phenyl-2,3-dihydro-1H-pyrazol-3-one
4-Dodecylbenzenesulfonic acid
Linoleic acid
NP-014113
NP-022389
NP-019159
NP-016724
NP-000124
4-Hydroxyindole
2-Furyl(5-hydroxy-1-benzofuran-3-yl)methanone
L-Aspartic acid-benzyl ester
D-Panthenol
[3-({3-[(Cyclopropylmethyl)amino]-3-oxetanyl}methyl)-1,2-oxazol-
5-yl]methanol
Stercobilin
DL-Carnitine
4-Indolecarbaldehyde
Thiamine
Lithocholic Acid
NP-009092
Biotin
4-(4-methylphenyl)-2-phenyl-1,3-thiazole
2-Morpholino-5-(1H-pyrrol-1-yl)benzoic acid
Disperse orange 3
N-(4-methoxy-1-methyl-1H-indazol-3-yl)-5-methyl-3-
isoxazolecarboxamide
L-Pyroglutamic acid
N-Acetyl-DL-glutamic acid
16-Hydroxyhexadecanoic acid
L-Saccharopine
Palmitelaidic acid methyl ester
9(Z),11(E),13(E)-Octadecatrienoic Acid methyl ester
D-Sphingosine
1-Linoleoyl glycerol
(6E)-7-(2H-1,3-benzodioxol-5-yl)-1-(piperidin-1-yl)hept-6-en-1-one
(2E,4E)-N-(2-methylpropyl)deca-2,4-dienamide
NP-016141
Piperine
Azelaic acid
Sebacic acid
Tropinone
2-Deoxyadenosine
Ethyl oleate
Salsinol
Lithocolic acid
N-Acetyl-L-tyrosine
Pipecolic acid
Pyridoxamine
N6-Acetyl-L-lysine
Riboflavin
O-Acetyl-L-carnitine
L-Aspartic acid

The disclosed bacteria include any bacteria of the genera, orders, or families of Table 2. Also disclosed are compositions comprising at least one bacteria from at least one genus, family, or order of Table 2.

TABLE 2
Bacteria

Actinomyces
Alistipes
Lachnospira
Gemella
Erysipelotrichaceae
Erysipelatoclostridium
Faecalibacterium
Family Lachnospiraceae (Lachnospiraceae_ND3007_group)
Family Oscillospiraceae (UCG_002)
Lacticaseibacillus
Methanobrevibacter
Order Coriobacteriales (Coriobacteriales_Incertae_Sedis uncultured)
Order Oscillospirales (Eubacterium_coprostanoligenes_group g)
Roseburia
Ruminococcus
Sellimonas
Streptococcus
Tyzzerella

Also disclosed are compositions comprising any metabolite disclosed herein and one or more bacteria disclosed herein. In some embodiments, the compositions comprises an indole metabolite and one or more bacteria disclosed herein. In some embodiments, the compositions comprises 4-hydroxyindole (4HI) and/or indole-4-carbaldehyde and one or more bacteria disclosed herein. The composition may specifically exclude any metabolite and/or bacteria disclosed herein.

### III. Therapeutic Compositions

Certain aspects relate to therapeutic compositions comprising a bacteria and/or a metabolite. The bacteria may be a strain of any of the bacteria listed in Table 2. In some aspects, the therapeutic composition comprises, consists of, or consists essentially of a bacteria in a unit dosage. The unit dosage may be any dosage sufficient for the desired effect of the therapeutic composition. In some aspects the unit dosage comprises between 1×10³ to 9×10¹⁶ colony forming units (CFU) of the bacteria. In some aspects the unit dosage comprises at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria. In another aspect, the disclosure relates to compositions comprising an isolated or purified population of any bacteria of Table 2. Moreover, a unit dosage may be given multiple times over a time period as discussed below.

Certain aspects relate to therapeutic compositions comprising a metabolite. The metabolite may be any metabolite, including a microbiota-derived metabolite. In certain aspects, the metabolite comprises an indole metabolite including 4HI and/or indole-4-carbaldehyde. In some aspects, the therapeutic composition comprises, consists of, or consists essentially of at least one metabolite in a unit dosage. The unit dosage may be any dosage sufficient for the desired effect of the therapeutic composition. In some aspects the unit dosage comprises between 1 to 999 μg, ng, mg, or g of the metabolite(s). In some aspects the unit dosage comprises at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, or 999 μg, ng, mg, g, μg/mL, ng/mL, mg/mL, g/mL, μg/kg, ng/kg, mg/kg, or g/kg, or any range derivable therein of the metabolite. Moreover, a unit dosage may be given multiple times over a time period as discussed below.

In some aspects, the therapeutic composition does not comprise a certain bacteria.

The therapeutic compositions can be formulated for administration, including as pharmaceutical formulations, e.g., formulated for oral administration; suppository administration; or injection such as via the intravenous, intramuscular, subcutaneous, or intraperitoneal routes. Such compositions can be prepared as either liquid solutions or suspensions; solid forms suitable for use to prepare solutions or suspensions upon the addition of a liquid prior to injection can also be prepared; and, the preparations can also be emulsified.

In certain aspects, the therapeutic composition is formulated for oral administration. The formulation for oral administration may comprise a pill, capsule, suspension, drink, or the like. In some aspects, the polysaccharide is administered through food.

In some aspects, the therapeutic composition is formulated for a fecal transplant. In some aspects, the fecal matter is administered in a dose of 50 g. In some embodiments, the fecal matter is administered in a dose of at least, at most, or exactly 10, 15, 20, 25, 30, 35, 40, 45, 50, 55, 60, 65, 70, 75, 80, 85, 90, 95, 100, 110, 120, 130, 140, 150, 160, 170, 180, 190, 200, 210, 220, 230, 240, 250, 260, 270, 280, 290, 300, 325, 350, 375, or 400 g (or any derivable range therein). In certain aspects, the fecal transplant comprises fecal matter that comprises a therapeutically effective amount of one or more bacteria of Table 2.

The pharmaceutical formulations suitable for injectable use include sterile aqueous solutions or dispersions; formulations including, for example, aqueous propylene glycol; and sterile powders for the extemporaneous preparation of sterile injectable solutions or dispersions. In certain aspects, the formulation is stable under the conditions of manufacture and storage and preserved against the contaminating action of non-therapeutic microorganisms.

A pharmaceutical composition or formulation can include a solvent or dispersion medium containing, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion, and by the use of surfactants. The prevention of the action of unintended microorganisms can be brought about by various anti-bacterial and anti-fungal agents, for example, parabens, chlorobutanol, phenol, sorbic acid, thimerosal, and the like. In certain aspects, the formulation includes isotonic agents, for example, sugars or sodium chloride. Prolonged absorption of the injectable compositions can be brought about by the use in the compositions of agents delaying absorption, for example, aluminum monostearate and gelatin.

Injectable solutions may be prepared by incorporating the active compounds in the required amount in the appropriate solvent with various other ingredients enumerated above, as required. In certain aspects encompassing powders for the preparation of injectable solutions, the therapeutic composition(s) are vacuum-dried and/or freeze-dried, which yield a powder of the active ingredient, plus any additional desired ingredient.

The present disclosure also provides a pharmaceutical composition comprising one or more microbial cultures as described above. The bacterial species therefore are present in the dose form as live bacteria, whether in dried, lyophilized, or sporulated form. This may be preferably adapted for suitable administration; for example, in tablet or powder form, potentially with an enteric coating, for oral treatment.

In particular aspects, the composition is formulated for oral administration. Oral administration may be achieved using a chewable formulation, a dissolving formulation, an encapsulated/coated formulation, a multi-layered lozenge (to separate active ingredients and/or active ingredients and excipients), a slow release/timed release formulation, or other suitable formulations known to persons skilled in the art. Although the word “tablet” is used herein, the formulation may take a variety of physical forms that may commonly be referred to by other terms, such as lozenge, pill, capsule, or the like.

While the compositions of the present disclosure can be formulated for oral administration, other routes of administration can be employed, however, including, but not limited to, intracolonic, subcutaneous, intramuscular, intradermal, transdermal, intraocular, intraperitoneal, mucosal, vaginal, rectal, and intravenous.

In another aspect, the disclosed composition may be prepared as a suppository. The suppository may include but is not limited to the bacteria, metabolites, and one or more carriers, such as polyethylene glycol, acacia, acetylated monoglycerides, carnuba wax, cellulose acetate phthalate, corn starch, dibutyl phthalate, docusate sodium, gelatin, glycerin, iron oxides, kaolin, lactose, magnesium stearate, methyl paraben, pharmaceutical glaze, povidone, propyl paraben, sodium benzoate, sorbitan monoleate, sucrose talc, titanium dioxide, white wax and coloring agents.

In some aspects, the composition may be prepared as a tablet. The tablet may include the bacteria, metabolites, and one or more tableting agents (i.e., carriers), such as dibasic calcium phosphate, stearic acid, croscarmellose, silica, cellulose and cellulose coating. The tablets may be formed using a direct compression process, though those skilled in the art will appreciate that various techniques may be used to form the tablets.

In other aspects, the composition may be formed as food or drink or, alternatively, as an additive to food or drink, wherein an appropriate quantity of bacteria and/or metabolite is added to the food or drink to render the food or drink the carrier.

The compositions of the present disclosure may further comprise one or more prebiotics known in the art, such as lactitol, inulin, or a combination thereof.

In some aspects, the composition may further comprise a food or a nutritional supplement effective to stimulate the growth of one or more bacteria present in Table 2 that are present in the gastrointestinal tract of the subject. In some aspects, the nutritional supplement is produced by a bacterium associated with a healthy human gut microbiome.

### IV. Administration of Therapeutic Compositions

Certain aspects concern the administration of therapies and therapeutic compositions, including any therapeutic composition described herein. The therapies may be administered in any suitable manner known in the art. The therapy provided herein may comprise administration of a combination of therapeutic composition, such as a first composition and a second composition. In some aspects, the first composition comprises any metabolite comprising composition described herein. In some aspects, the second composition comprises any bacterial composition described herein. The first and second compositions may be administered sequentially (at different times) or simultaneously (at the same time). In some aspects, the first and second compositions are administered as separate compositions. In some aspects, the first and second compositions are administered as the same composition.

In some aspects, the first therapeutic composition and the second therapeutic composition are administered substantially simultaneously. In some aspects, the first therapeutic composition and the second therapeutic composition are administered sequentially. In some aspects, the first therapeutic composition is administered before administering the second therapeutic composition. In some aspects, the first therapeutic composition is administered after administering the second therapeutic composition.

Aspects of the disclosure relate to compositions and methods comprising therapeutic compositions. The different therapies may be administered in one composition or in more than one composition, such as 2 compositions, 3 compositions, or 4 compositions. Various combinations of the agents may be employed.

In some aspects, the first composition, which may be a composition comprising a metabolite, and optionally the second composition, which may be a composition comprising a bacteria, are administered prophylactically. The first composition and optionally the second composition may be administered before the administration of a treatment that causes a disease or disorder, including endometriosis. In some aspects, the first composition and optionally the second composition are administered substantially simultaneously with another composition. In some aspects, the first composition and optionally the second composition are administered after the administration of another composition.

The therapeutic agents of the disclosure may be administered by the same route of administration or by different routes of administration. In some aspects, the therapeutic composition is administered intracolonically, intravenously, intramuscularly, subcutaneously, topically, orally, transdermally, intraperitoneally, intraorbitally, by implantation, by inhalation, intrathecally, intraventricularly, or intranasally. In some aspects, the antibiotic is administered intravenously, intramuscularly, subcutaneously, topically, orally, transdermally, intraperitoneally, intraorbitally, by implantation, by inhalation, intrathecally, intraventricularly, or intranasally. In some aspects, the composition is administered via gavage. The appropriate dosage may be determined based on the type of disease to be treated, severity and course of the disease, the clinical condition of the individual, the individual's clinical history and response to the treatment, and the discretion of the attending physician.

The treatments may include various “unit doses.” Unit dose is defined as containing a predetermined-quantity of the therapeutic composition. The quantity to be administered, and the particular route and formulation, is within the skill of determination of those in the clinical arts. A unit dose need not be administered as a single injection but may comprise continuous infusion over a set period of time. In some aspects, a unit dose comprises a single administrable dose.

In some aspects, a bacterial composition is administered. In some aspects, multiple doses of the bacterial composition are administered. In some aspects, the first therapeutic composition is administered at a dose of between 1×10⁵ to 9×10⁸ CFUs, or any range derivable therein. In some aspects, the first therapeutic composition is administered at a dose of at least, at most, or about 1×10³, 2×10³, 3×10³, 4×10³, 5×10³, 6×10³, 7×10³, 8×10³, 9×10³, 1×10⁴, 2×10⁴, 3×10⁴, 4×10⁴, 5×10⁴, 6×10⁴, 7×10⁴, 8×10⁴, 9×10⁴, 1×10⁵, 2×10⁵, 3×10⁵, 4×10⁵, 5×10⁵, 6×10⁵, 7×10⁵, 8×10⁵, 9×10⁵, 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, 9×10¹³, 1×10¹⁴, 2×10¹⁴, 3×10¹⁴, 4×10¹⁴, 5×10¹⁴, 6×10¹⁴, 7×10¹⁴, 8×10¹⁴, 9×10¹⁴, 1×10¹⁵, 2×10¹⁵, 3×10¹⁵, 4×10¹⁵, 5×10¹⁵, 6×10¹⁵, 7×10¹⁵, 8×10¹⁵, 9×10¹⁵, 1×10¹⁶, 2×10¹⁶, 3×10¹⁶, 4×10¹⁶, 5×10¹⁶, 6×10¹⁶, 7×10¹⁶, 8×10¹⁶, 9×10¹⁶, or any range derivable therein, CFU of the bacteria.

In some aspects, a single dose of the metabolite composition is administered. In some aspects, multiple doses of the metabolite composition are administered. In some aspects, the second therapeutic composition is administered at a dose of 1 μg/kg to 1 mg/kg, or any range derivable therein. In some aspects, the second therapeutic composition is administered at a dose of at least, at most, or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, or 999 μg, ng, mg, g, μg/mL, ng/ml, mg/mL, g/mL, μg/kg, ng/kg, mg/kg, or g/kg, or any range derivable therein.

The quantity to be administered, both according to number of treatments and unit dose, depends on the treatment effect desired. An effective dose is understood to refer to an amount necessary to achieve a particular effect.

Precise amounts of the therapeutic composition also depend on the judgment of the practitioner and are peculiar to each individual. Factors affecting dose include physical and clinical state of the individual, the route of administration, the intended goal of treatment (alleviation of symptoms versus cure) and the potency, stability and toxicity of the particular therapeutic substance or other therapies a subject may be undergoing.

It will be understood by those skilled in the art and made aware that dosage units such as μg/kg or mg/kg of body weight can be converted and expressed in comparable concentration units of μg/ml or mM (blood levels). It is also understood that uptake is species and organ/tissue dependent. The applicable conversion factors and physiological assumptions to be made concerning uptake and concentration measurement are well-known and would permit those of skill in the art to convert one concentration measurement to another and make reasonable comparisons and conclusions regarding the doses, efficacies and results described herein.

In certain instances, it will be desirable to have multiple administrations of the composition, e.g., 2, 3, 4, 5, 6 or more (and any range derivable therein) administrations. The administrations can be at 1, 2, 3, 4, 5, 6, 7, 8, to 5, 6, 7, 8, 9, 10, 11, 12, 13, 14 day, week, or month intervals, including all ranges there between.

The phrases “pharmaceutically acceptable” or “pharmacologically acceptable” refer to molecular entities and compositions that do not produce an adverse, allergic, or other untoward reaction when administered to an animal or human. As used herein, “pharmaceutically acceptable carrier” includes any and all solvents, dispersion media, coatings, anti-bacterial and anti-fungal agents, isotonic and absorption delaying agents, and the like. The use of such media and agents for pharmaceutical active substances is well known in the art. Except insofar as any conventional media or agent is incompatible with the active ingredients, its use in immunogenic and therapeutic compositions is contemplated. Supplementary active ingredients, such as other anti-infective agents and vaccines, can also be incorporated into the compositions.

Administration of the compositions will typically be via any common route. This includes, but is not limited to oral, suppository, or intravenous administration. Alternatively, administration may be by orthotopic, intradermal, subcutaneous, intramuscular, intraperitoneal, or intranasal administration. Such compositions would normally be administered as pharmaceutically acceptable compositions that include physiologically acceptable carriers, buffers or other excipients.

The desired dose of the composition of the present disclosure may be presented in multiple (e.g., two, three, four, five, six, or more) sub-doses administered at appropriate intervals throughout the day.

Upon formulation, solutions will be administered in a manner compatible with the dosage formulation and in such amount as is therapeutically or prophylactically effective. The formulations are easily administered in a variety of dosage forms, such as the type of injectable solutions described above.

### V. Methods of Treatment

Provided herein are methods for treating, delaying progression, and/or preventing certain diseases or disorders by administration of therapeutic compositions, such as compositions comprising metabolites and/or bacteria to a individual. In certain aspects, the disease or disorder comprises endometriosis. In some aspects, the treatment results in a sustained response, such as a sustained microbiome alteration and/or sustained reduction of endometriosis, in the individual after cessation of the treatment.

The term “treatment” or “treating” means any treatment of a disease in a mammal, including: (i) preventing the disease, that is, causing the clinical symptoms of the disease not to develop by administration of a protective composition prior to the induction of the disease; (ii) suppressing the disease, that is, causing the clinical symptoms of the disease not to develop by administration of a protective composition after the inductive event but prior to the clinical appearance or reappearance of the disease; (iii) inhibiting the disease, that is, arresting the development of clinical symptoms by administration of a protective composition after their initial appearance; and/or (iv) relieving the disease, that is, causing the regression of clinical symptoms by administration of a protective composition after their initial appearance. In some aspects, the treatment may exclude prevention of the disease.

In certain aspects, the individual administered a therapeutic composition is identified as having, or is at risk of having, a disease or disorder. The disease or disorder may be endometriosis. The disease or disorder may comprise the aberrant proliferation of endometriotic epithelial cells. The disease or disorder may comprise inflammation resulting from altered microbiota profiles.

In certain aspects, the administration of a therapeutic composition to a individual alters the microbiome, including altering the bacterial composition in the microbiome, and/or microbiome environment in the individual. In some aspects, the administration of the therapeutic composition increases the amount of at least one bacteria and/or bacteria and/or decreases the amount of at least one other bacteria and/or bacteria.

### VI. Methods of Determining Microbiome Composition

In some aspects, the methods relate to obtaining a microbiome profile of an individual. The microbiome profile may comprise bacteria and/or metabolites in the gastrointestinal tract of the individual. In some aspects, obtaining a microbiome profile comprises at least one of the steps, or the ordered steps, of: i) obtaining a sample obtained from a subject (e.g., a human subject), ii) isolating one or more bacterial species from the sample, iii) isolating one or more nucleic acids from at least one bacterial species, iv) sequencing the isolated nucleic acids, v) comparing the sequenced nucleic acids to a reference nucleic acid sequence, vi) isolating metabolites from the sample, vii) measuring the amounts of the isolated metabolites, and viii) comparing the measured metabolites to a reference. When performing the methods necessitating genotyping, any genotyping assay can be used. For example, this can be done by sequencing the 16S or the 23S ribosomal subunit or by metagenomics shotgun DNA sequencing associated with metatranscriptomics. When measuring metabolites, any metabolomics assay can be used, such as HPLC and/or mass spectrometry. In certain aspects, the levels of an indole metabolite, including 4HI and/or indole-4-carbaldehyde are measured to obtain the microbiome profile. In certain aspects, the levels of one or more metabolites of Table 1 are measured to obtain the microbiome profile. In certain aspects, the levels of one or more bacteria of Table 2 are measured to obtain the microbiome profile. In certain aspects, the levels of one or more metabolites of Table 1 and the levels of one or more bacteria of Table 2 are measured to obtain the microbiome profile.

In some aspects, obtaining the microbiome profile of an individual is used to monitor the need of administering therapeutic compositions, including any of the therapeutic compositions disclosed herein and/or additional therapeutic compositions, to the individual. In certain aspects, obtaining the microbiome profile of a individual is used to monitor the efficacy of the therapeutic compositions, including any therapeutic composition disclosed herein and/or additional therapeutic compositions, administered to the individual. In certain aspects, the individual is or is not administered a therapeutic composition based on the obtained microbiome profile of the individual. In certain aspects, the individual is administered a therapeutic composition because the obtained microbiome profile has an increased and/or decreased amount of one or more bacteria species and/or genus of bacteria when compared to a standard.

In certain aspects, the standard for comparison of the microbiome profile is a microbiome profile from a healthy individual, including an individual that does not, or has not been diagnosed with, endometriosis.

Methods for determining microbiome composition may include one or more microbiology methods such as sequencing, next generation sequencing, western blotting, comparative genomic hybridization, PCR, ELISA, metabolomics, etc.

In some aspects, the individual receiving a therapeutic composition, including any therapeutic composition described herein, has a higher abundance of at least one bacteria species and/or genus of bacteria.

Certain aspects relate to methods of prognosing the effectiveness of a therapy in a individual.

The methods can comprise contacting a biological sample, such as a stool sample, with one or more detection reagents. The detection reagents can be enzymes, antibodies, or other proteins capable of detecting specific metabolites, including one or more metabolites of Table 1. The detection reagents can be enzymes, antibodies, or other proteins capable of detecting specific bacteria, including any bacteria of Table 2. The detection reagents can be reagents to identify nucleic acid sequences specific to a genus, family, order, species, or strain of bacteria. The reagents can be primers, probes, and/or sequencing library preparation reagents. In some aspects, the nucleic acid from a biological sample is isolated prior to performing any of the methods described herein. In some aspects, polymerase chain reaction or other comparable techniques are used to amplify and/or detect specific sequences in the biological sample. The specific sequences may indicate the presence or absence of specific bacteria, including one or more bacteria of Table 2. Also contemplated are methods comprising measuring additional biomarkers, such as those known in the art as being associated with endometriosis, in addition to measuring the bacteria and/or metabolites disclosed herein.

The biological sample can be collected by the individual or by a healthcare provider. In some aspects, the biological sample is collected by the individual in a healthcare setting or outside of a healthcare setting. The biological sample may be contacted with buffers, preservatives, or other reagents to maintain the integrity of the biological sample after collection. The biological sample, which may be contacted with reagents to maintain the integrity of the biological sample, may be stored for 1, 2, 3, 4, 5, 6, 7, 8, 9, 10 or more days prior to measuring the bacteria and/or metabolites and/or performing any method steps described herein.

### VII. Kits

Certain aspects of the disclosure also encompass kits for performing the methods of the disclosure. Such kits can be prepared from readily available materials and reagents. For example, such kits can comprise any one or more of the following materials: enzymes, reaction tubes, buffers, detergent, primers, probes, antibodies.

In a particular aspect, these kits may comprise a plurality of agents for assessing or identifying microorganisms and/or metabolites, wherein the kit is housed in a container. The kits may further comprise instructions for using the kit for assessing sequences, means for converting and/or analyzing sequence data to generate a diagnosis and/or prognosis.

Kits may comprise a container with a label. Suitable containers include, for example, bottles, vials, and test tubes. The containers may be formed from a variety of materials such as glass or plastic. The container may hold a composition which includes a probe that is useful for prognostic or non-prognostic applications, such as described above. The label on the container may indicate that the composition is used for a specific prognostic or non-prognostic application, and may also indicate directions for either in vivo or in vitro use, such as those described above. The kit may comprise the container described above and one or more other containers comprising materials desirable from a commercial and user standpoint, including buffers, diluents, filters, needles, syringes, and package inserts with instructions for use.

Further kit aspects relate to kits comprising the therapeutic compositions of the disclosure. The kits may be useful in the treatment methods of the disclosure and comprise instructions for use.

### VIII. Microbiota Isolation and Culture

In some aspects, the metabolites are isolated and/or purified from a source. In some aspects, bacteria are isolated and/or purified from a source. The bacteria may be isolated and/or purified from any suitable source. In some aspects, the source is a human sample. In some aspects, the source is a non-human sample, such as a rodent sample. Bacteria and/or metabolites may be isolated and/or purified using any method known in the art. The isolated and/or purified bacteria and/or metabolites may then be formulated into therapeutic compositions, including the therapeutic compositions described herein. Before or after being isolated and/or purified, the bacteria may be characterized, including by sequencing to determine the composition and identity of the isolated and/or purified bacteria. The bacteria may be characterized by 16S rRNA or 23S rRNA sequencing.

In certain aspects, the methods described herein of treating a individual and methods comprising administering metabolites and/or bacteria to a individual further comprise isolating and/or purifying the metabolites and/or bacteria prior to the treatment and/or administration. The purified and/or isolated metabolites and/or bacteria may be used in the methods described herein.

In some aspects, the isolated and/or purified bacteria may be cultured for at least between about 1 days and about 40 days, for at least between about 5 days and about 35 days, for at least between about 5 days and 21 days. The bacteria may be cultured to generate sufficient quantity of bacteria to reach a unit dosage. The bacteria may be cultured for 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, or any range derivable therein, or more days. The bacteria may be cultured in the presence of a liquid culture medium, such as an LB broth or other suitable medium for culturing bacteria. Typically, the medium may comprise a basal medium formulation as known in the art. Compositions of the above basal media are generally known in the art, and it is within the skill of one in the art to modify or modulate concentrations of media and/or media supplements as necessary for the bacteria cultured. A defined medium, however, also can be used if the growth factors, cytokines, and hormones necessary for culturing the bacteria are provided at appropriate concentrations in the medium. Media useful in the methods of the disclosure may comprise one or more compounds of interest, including, but not limited to, antibiotics, mitogenic compounds, or differentiation compounds useful for the culturing of bacteria. The bacteria may be grown at temperatures between 27° C. to 40° C., such as 31° C. to 37° C., or any range derivable therein, and may be in a humidified incubator.

### EXAMPLES

The following examples are included to demonstrate preferred aspects of the disclosure. It should be appreciated by those of skill in the art that the techniques disclosed in the examples which follow represent techniques discovered by the inventor to function well in the practice of the disclosure, and thus can be considered to constitute preferred modes for its practice. However, those of skill in the art should, in light of the present disclosure, appreciate that many changes can be made in the specific aspects which are disclosed and still obtain a like or similar result without departing from the spirit and scope of the disclosure.

### Example 1

### Bacteria and Microbiota-Derived Metabolites Treating Endometriosis

Endometriosis is a debilitating health condition prevalent in reproductive age women, and that develops when endometrium (tissue lining the uterus) grows outside the uterus in some women due to reasons currently not understood. Inflammation resulting from these growing implants closely associates with disease severity and is aggravated by ‘leaky gut’ and has been linked to inflammatory bowel disorders. Several studies have addressed the concerns on perturbed gut microbiota profiles in endometriosis. However, no study reports the gut metabolome that mediate microbe-microbe and host-microbe interactions for pathogenesis. Here, the inventors compared metabolomic profiles of stool samples from patients with endometriosis (n=18) and healthy subjects (n=31). In total, the inventors derived 371 distinct metabolites using untargeted metabolomics and found several classes of metabolites (n=71) altered in endometriosis, mainly amino acids, carboxylic acids and nucleotide derivatives. The inventors identified biomarker metabolites from these global metabolomic profiles in endometriosis. The inventors also examined gut microbiome-wide associations of these metabolites through 16S rRNA gene profiling and catalogued robustly correlated pairs of bacterial taxa and metabolomic features. These perturbed metabolomic signatures profoundly resembled those in inflammatory bowel disease (IBD). Overall, the inventors discovered distinct bacteria-derived metabolites linked to endometriosis. Specifically, the inventors found two exclusively microbiota-derived derivatives of indole, namely, 4-hydroxyindole (4HI) and indole-4-carbaldehyde (I4CAld), that were significantly downregulated in endometriosis. In vitro assays confirmed their anti-proliferative effect on immortalized human endometriotic epithelial cells expressing luciferase (iHEECs/Luc). Administration of these metabolites in mice induced with endometriosis resulted in reduced lesion establishment and progression illustrating that these gut microbiota-derived metabolites of indole suppress the disease progression. The inventors further validated 4HI using LC-MS as the metabolite that is lower in stool samples of endometriosis. Notably, 4HI also reduced the endometriosis-associated hyperalgesia in mouse model of the disease. Overall, these findings can be used for non-invasive stool based diagnostic test for endometriosis, and 4-hydroxyindole (4HI) and indole-4-carbaldehyde (I4CAld) could be used for treating endometriosis.

All of the methods disclosed and claimed herein can be made and executed without undue experimentation in light of the present disclosure. While the compositions and methods of this disclosure have been described in terms of preferred aspects, it will be apparent to those of skill in the art that variations may be applied to the methods and in the steps or in the sequence of steps of the method described herein without departing from the concept, spirit and scope of the disclosure. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the disclosure as defined by the appended claims.

### REFERENCES

The following references, to the extent that they provide exemplary procedural or other details supplementary to those set forth herein, are specifically incorporated herein by reference.

What is claimed is:

1. A method of treating endometriosis in an individual, the method comprising administering to the individual an effective amount of at least one indole metabolite.

2. The method of claim 1, wherein the indole metabolite comprises 4-hydroxyindole (4HI) and/or indole-4-carbaldehyde.

3. The method of claim 2, wherein the indole metabolite is 4HI.

4. The method of claim 2, wherein the indole metabolite is indole-4-carbaldehyde.

5. The method of claim 2, wherein 4HI and indole-4-carbaldehyde are administered concurrently.

6. The method of claim 2, wherein 4HI and indole-4-carbaldehyde are administered sequentially.

7. The method of any one of claims 1-6, further comprising administering an additional therapeutic composition to the individual.

8. The method of any one of claims 1-7, wherein the individual is determined to have endometriosis after one or more metabolites and/or at least one bacteria is measured in a biological sample from the individual.

9. The method of claim 8, wherein the one or more metabolites comprise an indole metabolite.

10. The method of claim 9, wherein the indole metabolite comprises 4HI and/or indole-4-carbaldehyde.

11. The method of any one of claims 8-10, wherein the one or more metabolites comprise one or more metabolites from Table 1.

12. The method of any one of claims 8-11, wherein the bacteria is a bacteria from at least one bacterial genus, family, or order from Table 2.

13. The method of any one of claims 8-12, wherein the biological sample is a stool sample.

14. A method of treating endometriosis in an individual, the method comprising administering to the individual an effective amount of at least one metabolite of Table 1.

15. The method of claim 14, wherein the metabolite comprises 4-hydroxyindole (4HI) and/or indole-4-carbaldehyde.

16. The method of claim 15, wherein the metabolite is 4HI.

17. The method of claim 15, wherein the metabolite is indole-4-carbaldehyde.

18. The method of claim 15, wherein 4HI and indole-4-carbaldehyde are administered concurrently.

19. The method of claim 15, wherein 4HI and indole-4-carbaldehyde are administered sequentially.

20. The method of any one of claims 14-19, further comprising administering an additional therapeutic composition to the individual.

21. The method of any one of claims 14-20, wherein the individual is determined to have endometriosis after one or more metabolites and/or at least one bacteria is measured in a biological sample from the individual.

22. The method of claim 21, wherein the one or more metabolites comprise an indole metabolite.

23. The method of claim 22, wherein the indole metabolite comprises 4HI and/or indole-4-carbaldehyde.

24. The method of any one of claims 21-23, wherein the one or more metabolites comprise one or more metabolites from Table 1.

25. The method of any one of claims 21-24, wherein the bacteria is a bacteria from at least one bacterial genus, family, or order from Table 2.

26. The method of any one of claims 21-25, wherein the biological sample is a stool sample.

27. A therapeutic composition comprising 4-hydroxyindole (4HI) and indole-4-carbaldehyde.

28. The therapeutic composition of claim 27, further comprising an additional therapeutic composition.

29. A method for detecting endometriosis in an individual, the method comprising measuring one or more metabolites from Table 1 and/or at least one bacteria from Table 2 in a biological sample from the individual.

30. The method of claim 29, wherein the one or more metabolites comprise 4-hydroxyindole (4HI) and/or indole-4-carbaldehyde.

31. The method of claim 29 or 30, wherein the biological sample comprises a stool sample.

32. The method of any one of claims 29-31, wherein the method further comprises comparing the measured metabolites and/or measured bacteria to a standard.

33. The method of claim 32, wherein the standard comprises a measurement from a healthy individual.

34. The method of claim 33, wherein the measurement from the healthy individual is a measurement of the one or more metabolites from Table 1 and/or at least one bacteria from Table 2 in a biological sample from the healthy individual.

35. The method of claim 33 or 34, wherein the healthy individual is an individual known to not have endometriosis.

36. The method of any one of claims 29-35, further comprising administering to the individual an effective amount of at least one indole metabolite.

37. The method of claim 36, wherein the indole metabolite comprises 4-hydroxyindole (4HI) and/or indole-4-carbaldehyde.

38. The method of claim 37, wherein the indole metabolite is 4HI.

39. The method of claim 37, wherein the indole metabolite is indole-4-carbaldehyde.

40. The method of claim 37, wherein 4HI and indole-4-carbaldehyde are administered concurrently.

41. The method of claim 37, wherein 4HI and indole-4-carbaldehyde are administered sequentially.

42. The method of any one of claims 29-41, further comprising administering an effective amount of one or more metabolites of Table 1.

43. The method of any one of claims 29-42, further comprising administering an additional therapeutic composition to the individual.
